(19) United States
(12) Patent Application Publication
(10) Pub. No.: US 2003/0229935 A1
(43) Pub. Date: Dec. 18, 2003

(54) Land sport goggles

(76) Inventor: Terry Chou, Tainan City (CN)

Correspondence Address: Charles E. Baxley, 5th Floor, 59 John Street, New York, NY, 10038, US

(21) Appl. No.: 10/166,817

(22) Filed: Jun. 12, 2002

(51) Int. Cl. A61F 9/02

(52) U.S. Cl. 2/440

(57) ABSTRACT

A pair of goggles for land sports, the pair of goggles comprises a frame, at least one lens, and at least one padding member attached to an inner side of the frame. The padding member is made of soft, impermeable material. The padding member includes an engaging portion in an inner periphery thereof for engaging with the lens. The padding member includes a chamber for receiving air or fluid, thereby providing resiliency for absorbing shocks to prevent injury to a face of a wearer from the frame and the lens when the padding member is in intimate contact with the face of the wearer.

### BACKGROUND OF THE INVENTION

1. Field of the Invention

The present invention relates to a land sport goggles. In particular, the present invention relates to a pair of goggles having at least one padding member for providing improved intimate contact with the user's face for land sports.

2. Description of the Related Art

It is common to use a pair of goggles in land sports such as skiing, cycling, ball games, etc., to prevent injury to the eyes from strong wind or impingement. FIG. 7 of the drawings illustrates a pair of conventional goggles using a padding member 1′ made of sponge for intimate contact with the wearer's face. The padding member 1′ provides a shock-absorbing effect when subject to strong wind.

However, the padding member 1′ made of sponge has a limited resiliency such that the face of the wearer is still apt to be injured by the rigid lens and rigid frame of the goggles. Further, the overall appearance of the goggles is not aesthetic, as the padding member 1′ is made of sponge and the frame is generally made of plastics.

### SUMMARY OF THE INVENTION

An object of the present invention is to provide a pair of goggles having at least one padding member that is securely attached to an inner side of a frame of the goggles by any suitable means. The padding member is made of soft, impermeable material and includes an engaging portion in an inner periphery thereof for receiving a lens. The padding member includes a chamber for receiving air or fluid. When the padding member is compressed by an external force, a larger reactive force is obtained at the padding member due to provision of the air pressure or fluid pressure such that the wearer's face is kept away from the rigid frame and the rigid lens when the padding member is in intimate contact with the wearer's face.

The engaging portion of the padding member may be an engaging groove. At least one recessed portion is defined in an inner side of the padding member to provide a cushioning effect as well as a shock-absorbing effect. The padding member may be in the form of a ring. In an alternative embodiment, the padding member includes two half pieces separated by a gap for providing ventilation. The padding member may include a nose pad to allow easy assembly, to increase the wearing comfort, and to provide an overall good-looking appearance.

Other objects, advantages, and novel features of the invention will become more apparent from the following detailed description when taken in conjunction with the accompanying drawings.

### BRIEF DESCRIPTION OF THE DRAWINGS

FIG. 1 is an exploded perspective view of a pair of goggles in accordance with the present invention.

FIG. 2 is a perspective view of the pair of goggles in accordance with the present invention.

FIG. 3 is a schematic sectional view illustrating use of the pair of goggles in accordance with the present invention.

FIG. 4 is a schematic top view illustrating use of the pair of goggles in accordance with the present invention.

FIG. 5 is an exploded perspective view of a second embodiment of the pair of goggles in accordance with the present invention.

FIG. 6 is an exploded perspective view of a third embodiment of the pair of goggles in accordance with the present invention.

FIG. 7 is a schematic sectional view illustrating use of a pair of conventional goggles.

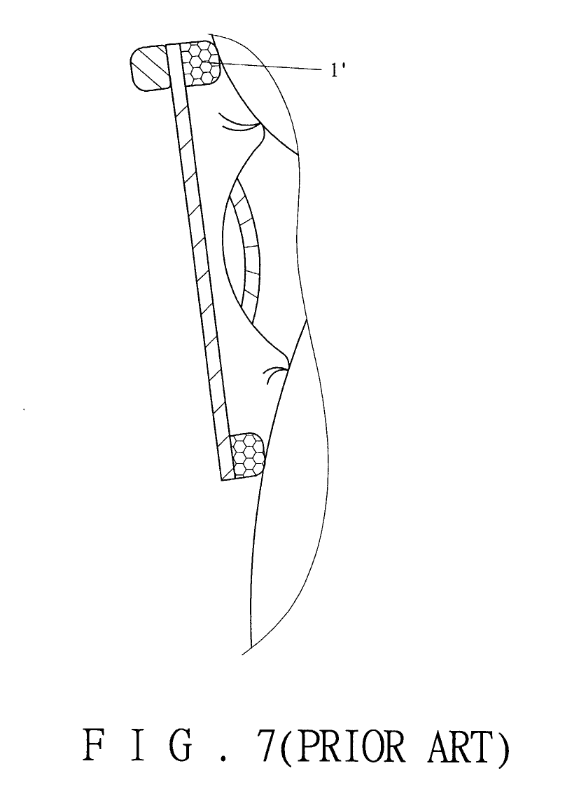

### DETAILED DESCRIPTION OF THE PREFERRED EMBODIMENTS

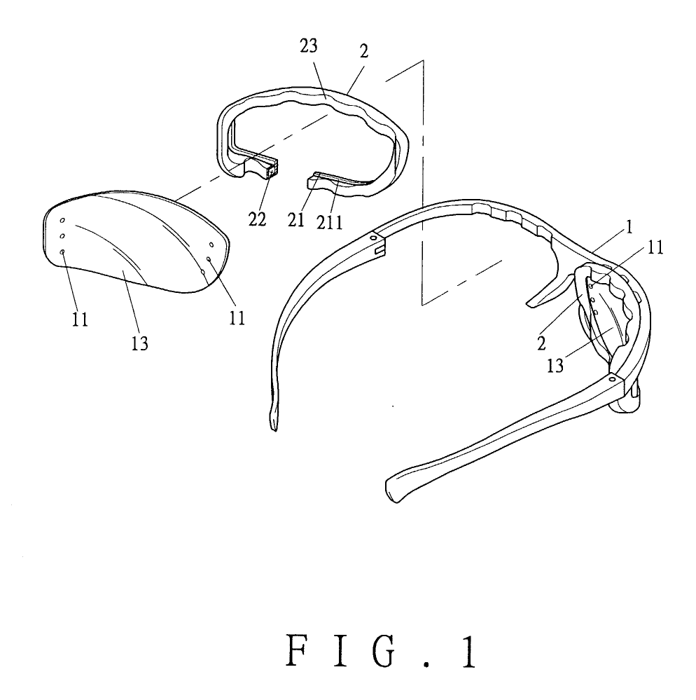

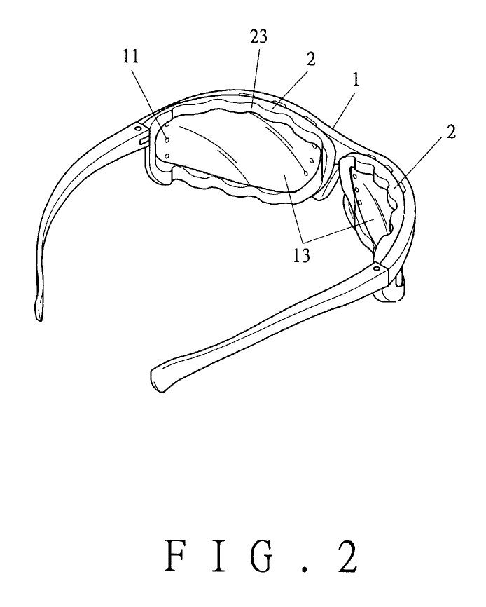

Referring to FIGS. 1 through 3, a pair of land sport goggles in accordance with the present invention generally comprises a frame 1 and at least one padding member 2 attached to an inner side of the frame 1 by any suitable means, such as male and female engagement, bonding, etc.

In this embodiment, there are two ring-like padding members 2 attached to the inner side of the frame 1. Each padding member 2 is made of soft, impermeable material and includes an engaging portion 21 in an inner periphery thereof. An engaging groove 211 is defined in the engaging portion 21 for receiving a lens 13 through stretchability of the padding member 2. Further, each padding member 2 includes a chamber 22 along a length thereof. Air or fluid may be filled into the chamber 22. Further, at least one recessed portion 23 may be formed on an inner side of each padding member 2 for ventilation as well as providing improved resiliency for absorbing shock. At least one vent 11 may be provided in each lens 13 or the frame 1 to assist in ventilation.

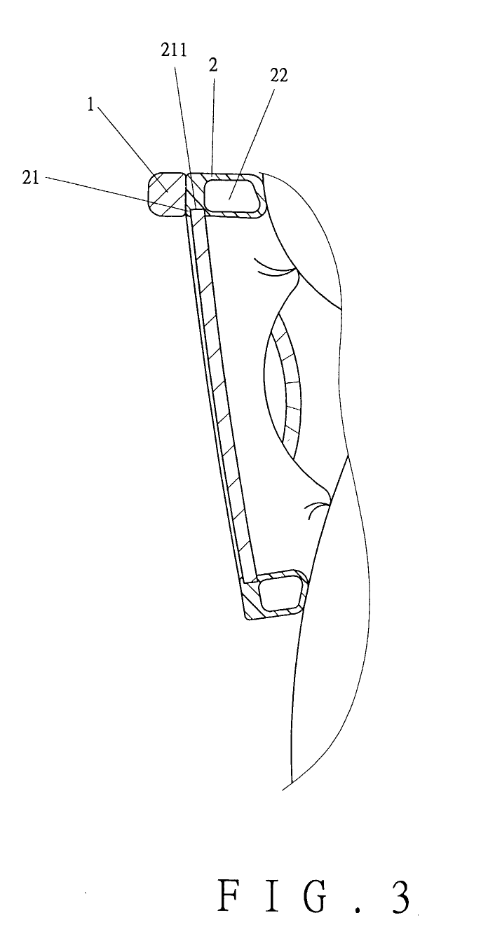

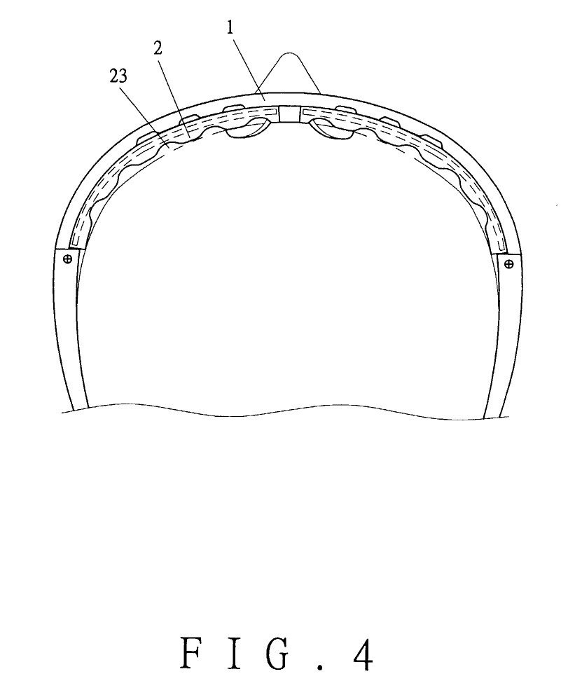

Referring to FIGS. 3 and 4, when in use, each padding member 2 is in intimate contact with the wearer's face. The channel 22 of each padding member 2 provides a shock-absorbing function to prevent injury to the wearer from the rigid frame 1 and the rigid lenses 13. When the padding member 2 is compressed by an external force, a larger reactive force is obtained at the padding member 2 due to provision of the air pressure or fluid pressure such that the wearer's face is kept away from the rigid frame 1 and the rigid lenses 13 when the padding member is in intimate contact with the wearer's face. The recessed portions 23 also provide a cushioning effect as well as a shock-absorbing effect.

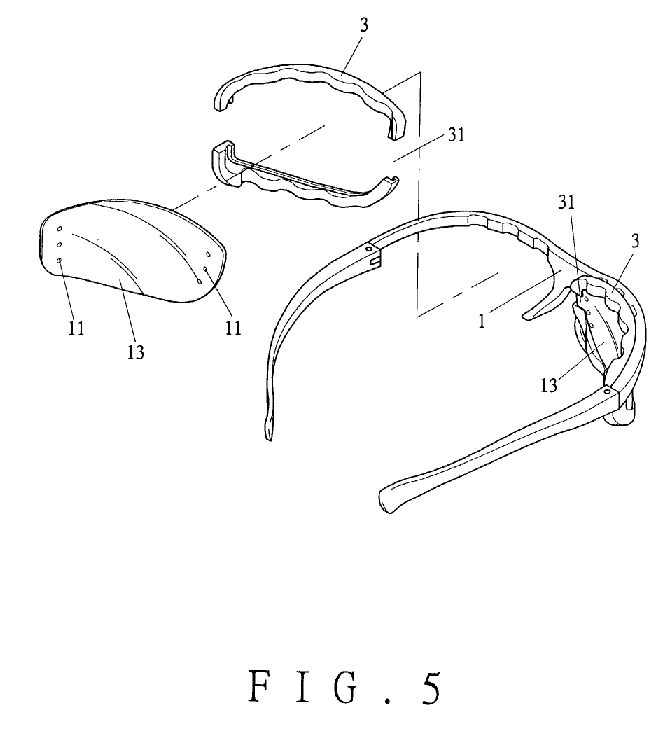

FIG. 5 illustrates a second embodiment of the padding member. In this embodiment, the padding member (now designated by 3) includes two half pieces separated by a gap 31 for providing ventilation.

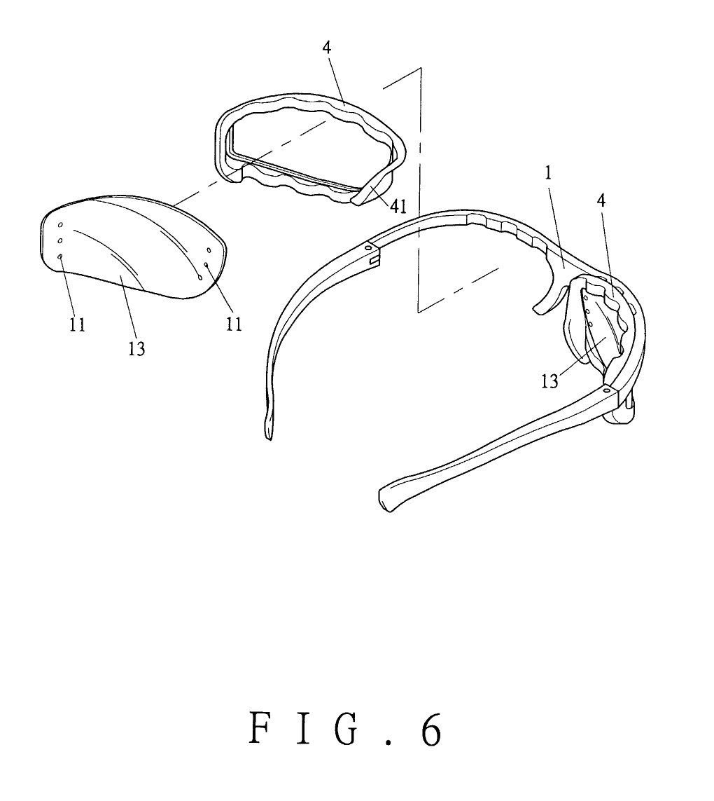

FIG. 6 illustrates a third embodiment of the padding member. In this embodiment, the padding member (now designated by 4) includes a nose pad 41 for contact with the wearer's nose. Of course, the nose pad 41 may include a chamber similar to the chamber 22 in the first embodiment for receiving air or fluid, thereby improving the shock-absorbing effect. Further, the assembly of the goggles is improved, and the overall appearance is good looking.

According to the above description, it is appreciated that the goggles in accordance with the present invention has many advantages when compared with conventional goggles. Firstly, the air or fluid in the chamber 22 of the padding member 2 improves the shock-absorbing effect and prevents injury to the wearer' face from the rigid portions of the goggles. Second, the recessed portions 23 on the padding member 2 provide ventilation even if the lens 13 or the frame 1 is not provided with vents 11. Thus, the padding member 2 can be used with any type of frame 1 while providing improved ventilation. Third, the engagement between the engaging groove 211 of the padding member 2 and the lens 1 allows easy assembly, as no boding agent is required. Fourth, the padding member 4 may include a nose pad 41 to improve the shock-absorbing effect in the nose area of the wearer, and the nose portion of the frame 1 may be omitted. The cost of manufacture is reduced, and an aesthetic appearance of the goggles is provided. Fifth, the padding member 4 can be made of plastic material similar to that for the frame 1, further improving the overall appearance.

Although the invention has been explained in relation to its preferred embodiments, it is to be understood that many other possible modifications and variations can be made without departing from the scope of the invention as hereinafter claimed.

What is claimed is:

1. A pair of goggles for land sports, the pair of goggles comprising:
a frame;
at least one lens; and
at least one padding member attached to an inner side of the frame, said at least one padding member being made of soft, impermeable material, said at least one padding member including an engaging portion in an inner periphery thereof for engaging with said at least one lens, said at least one padding member including a chamber for receiving air or fluid, thereby providing resiliency for absorbing shocks to prevent injury to a face of a wearer from the frame and said at least one lens when said at least one padding member is in intimate contact with the face of the wearer.

2. The pair of goggles for land sports as claimed in claim 1, wherein said at least one padding member includes at least one recessed portion in an inner side thereof for providing ventilation and shock-absorbing effect.

3. The pair of goggles for land sports as claimed in claim 1, wherein said at least one padding member is a ring.

4. The pair of goggles for land sports as claimed in claim 1, wherein said at least one padding member includes two half pieces separated by a gap for providing ventilation.

5. The pair of goggles for land sports as claimed in claim 1, wherein said at least one padding member, wherein the engaging portion of said at least one padding member includes an engaging groove for receiving said at least one lens through stretchability of said at least one padding member.

6. The pair of goggles for land sports as claimed in claim 1, wherein said at least one padding member is attached to said frame by bonding.

7. The pair of goggles for land sports as claimed in claim 1, wherein said at least one padding member includes a nose pad for contact with a nose of the wearer.
